(19) United States
(12) Patent Application Publication
(10) Pub. No.: US 2022/0022821 A1
(43) Pub. Date: Jan. 27, 2022

(54) ANONYMOUS CONTACT TRACING SYSTEM AND METHOD

(71) Applicant: TATO LLC, Bloomington, IN (US)

(72) Inventor: Peter James Rodgers, Bloomington, IN (US)

(73) Assignee: TATO LLC, Bloomington, IN (US)

(21) Appl. No.: 17/383,148

(22) Filed: Jul. 22, 2021

Related U.S. Application Data
(60) Provisional application No. 63/103,192, filed on Jul. 24, 2020.

(51) Int. Cl. A61B 5/00; G16H 50/80; H04L 12/18; H04W 4/029; H04W 4/021

(52) U.S. Cl. CPC A61B 5/746; G16H 50/80; G16H 50/70; H04W 4/029; H04W 4/021; H04L 12/1895

(57) ABSTRACT

The present invention involves a method for anonymous contact tracing for a user with a mobile device in a location having an associated contract tracing system having a plurality of tokens. The user has a mobile device upon which the user obtains a token from the location and/or the contact tracing system. The mobile device stores the user's space-time coordinate information. There is a recording of space-time coordinate information of each mobile device that is traceable by the contact tracing system in association with one of the tokens. When a user reports the existence of an exposure event, the contact tracing system analyzes the token-coordinate information to determine which of the tokens have been subject to the exposure event. By notifying at least one of the users with an associated token that was subject to the exposure event, those users may also check to see if they have been subject to the same exposure event. The method may be implemented by a server and by software running on a mobile device. The present invention involves an anonymous contact tracing system and method for a user with a mobile device in a location having a contract tracing system. The method steps include obtaining a token from the contact tracing system using a mobile device; storing the user's space-time coordinate information on the mobile device; recording space-time coordinate information of each mobile device that is traceable by the contact tracing system in association with the associated tokens; reporting the existence of an exposure event by the user; analyzing all of the contact tracing system's token-coordinate information to determine tokens that have been subject to the exposure event; and notifying at least one of the users having an associated token having been subject to the exposure event. In addition, the contact tracing system may include a website. The step of obtaining may be performed by the mobile device requesting a token from the contract tracing system website. The token is uniquely identifiable by the contact tracing system, and lacks any personally identifying information relating to the user, and the obtained token may be stored with the user's space-time coordinate information, optionally in a browser storage of the mobile device. The invention further involves a server for implementing the method, and a mobile device App enabling the method.

### CROSS-REFERENCE TO RELATED APPLICATIONS

The present application claims priority under 35 U.S.C. § 119(e) of U.S. Patent Provisional Application Ser. No. 63/103,192, filed Jul. 24, 2020, assigned to the assignee of the present application, the disclosures of which are incorporated by reference herein.

### BACKGROUND OF THE INVENTION

### Field of the Invention

The invention relates to contact tracing software and systems. More specifically, but not exclusively, the field of the invention is that of contact tracing software and systems for individuals wishing to maintain anonymity.

### Description of the Related Art

In times of pandemic or other public health emergency it is important, in the event that a physical location has been exposed to an infectious agent, that people who were present at the location at that time should be notified of their potential risk of exposure—so called contact tracing.

Numerous implementations of contact tracing solutions exist taking the form of mobile phone apps that employ various mechanisms such as bluetooth and/or WiFi MAC address logging, continuous GPS geo-location tracking etc. In each case considerable concern has been raised about loss of anonymity, the continuous surveillance necessary to afford a sufficient data set and the requirement that the space-time record is uploaded for aggregation in a centralized database. What is lacking in the art is such a system that allows users anonymity.

### SUMMARY OF THE INVENTION

The present invention is a method and system for anonymous contact tracing which allows user anonymity. According to embodiments of the invention, standard web technologies are used in which the space-time contact tracing data remains the property of a user and is only shared in the event of a user desiring to report notice of infection to a location, or, in the event that a location has a notice of infection and has a duty of care to inform people that may have been exposed. At all times the identity of the user is anonymous to the location. When used with cloud computing, the cloud service that facilitates the solution also does not possess knowledge of the user. By employing standard web technologies the necessity to install a special purpose mobile phone app is eliminated ensuring universal compatibility and eliminating the barrier to adoption. Most important this also removes the loss of anonymity that is implicit when installing an app from an app store.

The present invention, in another form, is a method for anonymous contact tracing. First, the user to obtain a token from a location's website using a mobile device. The token is unique and identifiable by the location's software for future tracing, but lacks any personally identifying information. On the user's mobile device, the user's space-time information is stored, optionally in the same location as the token. Next, the location maintains records of the space-time coordinates of each token while the mobile devices that were given the token are located in the location. The user may report the existence of an exposure event (for example, interacting with an individual having tested positive for a virus, or entering an area with an abnormal concentration of mold or spores) to the location using the user's token and a description of the exposure event. Lastly, the location software may analyze all of its token-coordinate information to determine all the tokens that have been subject to the exposure event. All token holders may then be sent a warning message indicating the existence of the exposure event. In one embodiment, the warning message may include an indication of all effected token holders, and the mobile device through the website or the app would check its token value against the token identifications. In another embodiment, the warning message may only indicate that an exposure event occurred and the user device would have the option to check to see if the stored token was one of the identified exposed tokens.

Embodiments of the invention perform a method of anonymous contact tracing for a user with a mobile device in a location having a contract tracing system. The method steps include obtaining a token from the contact tracing system using a mobile device; storing the user's space-time coordinate information on the mobile device; recording space-time coordinate information of each mobile device that is traceable by the contact tracing system in association with the associated tokens; reporting the existence of an exposure event by the user; analyzing all of the contact tracing system's token-coordinate information to determine tokens that have been subject to the exposure event; and notifying at least one of the users having an associated token having been subject to the exposure event. In addition, the contact tracing system may include a website. The step of obtaining may be performed by the mobile device requesting a token from the contract tracing system website. The token is uniquely identifiable by the contact tracing system, and lacks any personally identifying information relating to the user, and the obtained token may be stored with the user's space-time coordinate information, optionally in a browser storage of the mobile device. To report an exposure event, the user may identifying the user's token and provide a description of the exposure event. Once reported, the notifying step may include sending a message with an indication of all effected token holders. The user's mobile device may then check the mobile device's stored token value against the token identifications of the message. Alternatively, the notifying step may include sending a warning message indicating that an exposure event occurred, and the user's mobile device may then check the contact tracing system to determine if the stored token was one of the identified exposed tokens.

Further aspects of the present invention involve a server for implementing the previously described method.

Another aspect of the invention relates to an App for a mobile device for performing a method of anonymous contact tracing according to the foregoing method.

### BRIEF DESCRIPTION OF THE DRAWINGS

The above mentioned and other features and objects of this invention, either alone or in combinations of two or more, and the manner of attaining them, will become more apparent and the invention itself will be better understood by reference to the following description of an embodiment of the invention taken in conjunction with the accompanying drawings, wherein:

FIG. 1 is a schematic diagrammatic view of a network system in which embodiments of the present invention may be utilized.

FIG. 2 is a block diagram of a computing system (either a server or client, or both, as appropriate), with optional input devices (e.g., keyboard, mouse, touch screen, etc.) and output devices, hardware, network connections, one or more processors, and memory/storage for data and modules, etc. which may be utilized in conjunction with embodiments of the present invention.

FIG. 3 is a flow chart diagram of the operation of the present invention relating to the process steps of one embodiment of the invention.

Corresponding reference characters indicate corresponding parts throughout the several views. Although the drawings represent embodiments of the present invention, the drawings are not necessarily to scale and certain features may be exaggerated in order to better illustrate and explain the full scope of the present invention. The flow charts and screen shots are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

### DESCRIPTION OF EMBODIMENTS OF THE PRESENT INVENTION

The embodiment disclosed below is not intended to be exhaustive or limit the invention to the precise form disclosed in the following detailed description. Rather, the embodiment is chosen and described so that others skilled in the art may utilize its teachings. While technology should continue to develop and many of the elements of the embodiments disclosed may be replaced by improved and enhanced items, the teaching of the present invention are inherent in the disclosure of the elements used in embodiments using technology available at the time of this disclosure.

The detailed descriptions which follow are presented in part in terms of algorithms and symbolic representations of operations on data bits within a computer memory representing alphanumeric characters or other information. A computer generally includes a processor for executing instructions and memory for storing instructions and data. When a general purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the art of data processing arts to most effectively convey the substance of their work to others skilled in the art.

An algorithm is here, and generally, conceived to be a self-consistent sequence of steps leading to a desired result. These steps are those requiring physical manipulations of physical quantities. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed. It should be borne in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through sophisticated software systems. Data structures are not the information content of a memory, rather they represent specific electronic structural elements which impart or manifest a physical organization on the information stored in memory. More than mere abstraction, the data structures are specific electrical or magnetic structural elements in memory which simultaneously represent complex data accurately, often data modeling physical characteristics of related items, and provide increased efficiency in computer operation. By changing the organization and operation of data structures and the algorithms for manipulating data in such structures, the fundamental operation of the computing system may be changed and improved.

Further, the manipulations performed are often referred to in terms, such as comparing or adding, commonly associated with mental operations performed by a human operator. No such capability of a human operator is necessary, or desirable in most cases, in any of the operations described herein which form part of embodiments of the present invention; the operations are machine operations. The operations of these algorithms are deterministic with the accuracy and complexity management that are not obtainable by human mental steps even though the language used to describe them in the detailed description below at some time references a mental step. This requirement for machine implementation for the practical application of the algorithms is understood by those persons of skill in this art as not a duplication of human thought, rather as significantly more than such duplication. Useful machines for performing the operations of one or more embodiments of the present invention include general purpose digital computers or other similar devices. In all cases the distinction between the method operations in operating a computer and the method of computation itself should be recognized. One or more embodiments of present invention relate to methods and apparatus for operating a computer in processing electrical or other (e.g., mechanical, chemical) physical signals to generate other desired physical manifestations or signals. The computer operates on software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level coding of the instructions that is interpreted to obtain the actual computer code. The software module may also include a hardware component, wherein some aspects of the algorithm are performed by the circuitry itself rather as a result of an instruction.

Some embodiments of the present invention also relate to an apparatus for performing these operations. This apparatus may be specifically constructed for the required purposes or it may comprise a general purpose computer as selectively activated or reconfigured by a computer program stored in the computer. The algorithms presented herein are not inherently related to any particular computer or other apparatus unless explicitly indicated as requiring particular hardware. In some cases, the computer programs may communicate or relate to other programs or equipment through signals configured to particular protocols which may or may not require specific hardware or programming to interact. In particular, various general-purpose machines may be used with programs written in accordance with the teachings herein, or it may prove more convenient to construct more specialized apparatus to perform the required method steps. The required structure for a variety of these machines will appear from the description below.

Embodiments of the present invention may deal with “object-oriented” software, and particularly with an “object-oriented” operating system. The “object-oriented” software is organized into “objects”, each comprising a block of computer instructions describing various procedures (“methods”) to be performed in response to “messages” sent to the object or “events” which occur with the object. Such operations include, for example, the manipulation of variables, the activation of an object by an external event, and the transmission of one or more messages to other objects.

Messages are sent and received between objects having certain functions and knowledge to carry out processes. Messages are generated in response to user instructions, for example, by a user activating an icon with a “mouse” pointer generating an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and where the other objects are not allowed to access. One feature of the object-oriented system is inheritance. For example, an object for drawing a “circle” on a display may inherit functions and knowledge from another object for drawing a “shape” on a display.

A programmer “programs” in an object-oriented programming language by writing individual blocks of code each of which creates an object by defining its methods. A collection of such objects adapted to communicate with one another by means of messages comprises an object-oriented program. Object-oriented computer programming facilitates the modeling of interactive systems in that each component of the system may be modeled with an object, the behavior of each component being simulated by the methods of its corresponding object, and the interactions between components being simulated by messages transmitted between objects.

An operator may stimulate a collection of interrelated objects comprising an object-oriented program by sending a message to one of the objects. The receipt of the message may cause the object to respond by carrying out predetermined functions which may include sending additional messages to one or more other objects. The other objects may in turn carry out additional functions in response to the messages they receive, including sending still more messages. In this manner, sequences of message and response may continue indefinitely or may come to an end when all messages have been responded to and no new messages are being sent. When modeling systems utilizing an object-oriented language, a programmer need only think in terms of how each component of a modeled system responds to a stimulus and not in terms of the sequence of operations to be performed in response to some stimulus. Such sequence of operations naturally flows out of the interactions between the objects in response to the stimulus and need not be preordained by the programmer.

Although object-oriented programming makes simulation of systems of interrelated components more intuitive, the operation of an object-oriented program is often difficult to understand because the sequence of operations carried out by an object-oriented program is usually not immediately apparent from a software listing as in the case for sequentially organized programs. Nor is it easy to determine how an object-oriented program works through observation of the readily apparent manifestations of its operation. Most of the operations carried out by a computer in response to a program are “invisible” to an observer since only a relatively few steps in a program typically produce an observable computer output.

In the following description, several terms which are used frequently have specialized meanings in the present context. The term “object” relates to a set of computer instructions and associated data which may be activated directly or indirectly by the user. The terms “windowing environment”, “running in windows”, and “object oriented operating system” are used to denote a computer user interface in which information is manipulated and displayed on a video display such as within bounded regions on a raster scanned, liquid crystal matrix, or plasma based video display (or any similar type video display that may be developed). The terms “network”, “local area network”, “LAN”, “wide area network”, or “WAN” mean two or more computers which are connected in such a manner that messages may be transmitted between the computers. In such computer networks, typically one or more computers operate as a “server”, a computer with large storage devices such as hard disk drives and communication hardware to operate peripheral devices such as printers or modems. Other computers, termed “workstations”, provide a user interface so that users of computer networks may access the network resources, such as shared data files, common peripheral devices, and inter-workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis. A “module” refers to a portion of a computer system and/or software program that carries out one or more specific functions and may be used alone or combined with other modules of the same system or program.

The term “desktop” means a specific user interface which presents a menu or display of objects with associated settings for the user associated with the desktop. When the desktop accesses a network resource, which typically requires an application program to execute on the remote server, the desktop calls an Application Program Interface, or “API”, to allow the user to provide commands to the network resource and observe any output. The term “Browser” refers to a program which is not necessarily apparent to the user, but which is responsible for transmitting messages between the desktop or a mobile device and the network server and for displaying and interacting with the network user. Browsers are designed to utilize a communications protocol for transmission of text and graphic information over a world wide network of computers, namely the “World Wide Web” or simply the “Web”. Examples of Browsers compatible with one or more embodiments of the present invention include the Chrome browser program developed by Google Inc. of Mountain View, Calif. (Chrome is a trademark of Google Inc.), the Safari browser program developed by Apple Inc. of Cupertino, Calif. (Safari is a registered trademark of Apple Inc.), Internet Explorer program developed by Microsoft Corporation (Internet Explorer is a trademark of Microsoft Corporation), the Opera browser program created by Opera Software ASA, or the Firefox browser program distributed by the Mozilla Foundation (Firefox is a registered trademark of the Mozilla Foundation). Although the following description details such operations in terms of a graphic user interface of a Browser, one or more embodiments of the present invention may be practiced with text based interfaces, or even with voice or visually activated interfaces, that have many of the functions of a graphic based Browser.

Browsers display information which is formatted in a Standard Generalized Markup Language (“SGML”) or a HyperText Markup Language (“HTML”), both being scripting languages which embed non-visual codes in a text document through the use of special ASCII text codes. Files in these formats may be easily transmitted across computer networks, including global information networks like the Internet, and allow the Browsers to display text, images, and play audio and video recordings. The Web utilizes these data file formats to conjunction with its communication protocol to transmit such information between servers and workstations. Browsers may also be programmed to display information provided in an eXtensible Markup Language (“XML”) file, with XML files being capable of use with several Document Type Definitions (“DTD”) and thus more general in nature than SGML or HTML. The XML file may be analogized to an object, as the data and the stylesheet formatting are separately contained (formatting may be thought of as methods of displaying information, thus an XML file has data and an associated method). Similarly, JavaScript Object Notation (JSON) may be used to convert between data file formats.

The terms “personal digital assistant”, or “PDA”, or smartphone as defined above, means any handheld, mobile device that combines two or more of computing, telephone, fax, e-mail and networking features. The terms “wireless wide area network” or “WWAN” mean a wireless network that serves as the medium for the transmission of data between a handheld device and a computer. The term “synchronization” means the exchanging of information between a first device, e.g. a handheld device, and a second device, e.g. a desktop computer or a computer network, either via wires or wirelessly. Synchronization ensures that the data on both devices are identical (at least at the time of synchronization).

Data may also be synchronized between computer systems and telephony systems. Such systems are known and include keypad based data entry over a telephone line, voice recognition over a telephone line, and voice over internet protocol (“VoIP”). In this way, computer systems may recognize callers by associating particular numbers with known identities. More sophisticated call center software systems integrate computer information processing and telephony exchanges. Such systems initially were based on fixed wired telephony connections, but such systems have migrated to wireless technology.

In wireless wide area networks, communication primarily occurs through the transmission of radio signals over analog, digital cellular or personal communications service (“PCS”) networks. Signals may also be transmitted through microwaves and other electromagnetic waves. Much wireless data communication takes place across cellular systems using second generation technology such as code-division multiple access (“CDMA”), time division multiple access (“TDMA”), the Global System for Mobile Communications (“GSM”), Third Generation (wideband or “3G”), Fourth Generation (broadband or “4G”), personal digital cellular (“PDC”), or through packet-data technology over analog systems such as cellular digital packet data (“CDPD”) used on the Advance Mobile Phone Service (“AMPS”).

The terms “wireless application protocol” or “WAP” mean a universal specification to facilitate the delivery and presentation of web-based data on handheld and mobile devices with small user interfaces. “Mobile Software” refers to the software operating system which allows for application programs to be implemented on a mobile device such as a mobile telephone or PDA. Examples of Mobile Software are Java and Java ME (Java and JavaME are trademarks of Sun Microsystems, Inc. of Santa Clara, Calif.), BREW (BREW is a registered trademark of Qualcomm Incorporated of San Diego, Calif.), Windows Mobile (Windows is a registered trademark of Microsoft Corporation of Redmond, Wash.), Palm OS (Palm is a registered trademark of Palm, Inc. of Sunnyvale, Calif.), Symbian OS (Symbian is a registered trademark of Symbian Software Limited Corporation of London, United Kingdom), ANDROID OS (ANDROID is a registered trademark of Google, Inc. of Mountain View, Calif.), and iPhone OS (iPhone is a registered trademark of Apple, Inc. of Cupertino, Calif.), and Windows Phone 7. “Mobile Apps” refers to software programs written for execution with Mobile Software.

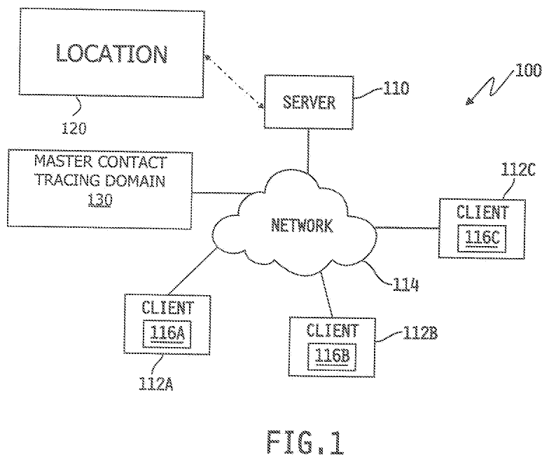

FIG. 1 is a high-level block diagram of a computing environment 100 according to one embodiment. FIG. 1 illustrates server 110 and three clients 112 connected by network 114. Only three clients 112 are shown in FIG. 1 in order to simplify and clarify the description. Embodiments of the computing environment 100 may have thousands or millions of clients 112 connected to network 114, for example the Internet. Users (not shown) may operate software 116 on one of clients 112 to both send and receive messages network 114 via server 110 and its associated communications equipment and software (not shown).

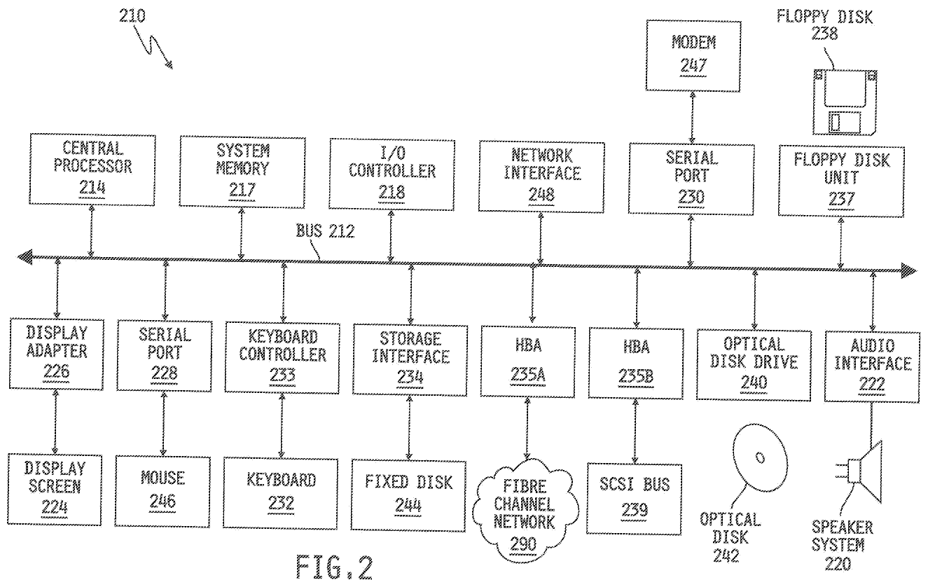

FIG. 2 depicts a block diagram of computer system 210 suitable for implementing server 110 or client 112. Computer system 210 includes bus 212 which interconnects major subsystems of computer system 210, such as central processor 214, system memory 217 (typically RAM, but which may also include ROM, flash RAM, or the like), input/output controller 218, external audio device, such as speaker system 220 via audio output interface 222, external device, such as display screen 224 via display adapter 226, serial ports 228 and 230, keyboard 232 (interfaced with keyboard controller 233), storage interface 234, disk drive 237 operative to receive floppy disk 238 (disk drive 237 is used to represent various type of removable memory such as flash drives, memory sticks and the like), host bus adapter (HBA) interface card 235A operative to connect with Fibre Channel network 290, host bus adapter (HBA) interface card 235B operative to connect to SCSI bus 239, and optical disk drive 240 operative to receive optical disk 242. Also included are mouse 246 (or other point-and-click device, coupled to bus 212 via serial port 228), modem 247 (coupled to bus 212 via serial port 230), and network interface 248 (coupled directly to bus 212).

Bus 212 allows data communication between central processor 214 and system memory 217, which may include read-only memory (ROM) or flash memory (neither shown), and random access memory (RAM) (not shown), as previously noted. RAM is generally the main memory into which operating system and application programs are loaded. ROM or flash memory may contain, among other software code, Basic Input-Output system (BIOS) which controls basic hardware operation such as interaction with peripheral components. Applications resident with computer system 210 are generally stored on and accessed via computer readable media, such as hard disk drives (e.g., fixed disk 244), optical drives (e.g., optical drive 240), floppy disk unit 237, or other storage medium. Additionally, applications may be in the form of electronic signals modulated in accordance with the application and data communication technology when accessed via network modem 247 or interface 248 or other telecommunications equipment (not shown).

Storage interface 234, as with other storage interfaces of computer system 210, may connect to standard computer readable media for storage and/or retrieval of information, such as fixed disk drive 244. Fixed disk drive 244 may be part of computer system 210 or may be separate and accessed through other interface systems. Modem 247 may provide direct connection to remote servers via telephone link or the Internet via an internet service provider (ISP) (not shown). Network interface 248 may provide direct connection to remote servers via direct network link to the Internet via a POP (point of presence). Network interface 248 may provide such connection using wireless techniques, including digital cellular telephone connection, Cellular Digital Packet Data (CDPD) connection, digital satellite data connection or the like.

Many other devices or subsystems (not shown) may be connected in a similar manner (e.g., document scanners, digital cameras and so on). Conversely, all of the devices shown in FIG. 2 need not be present to practice the present disclosure. Devices and subsystems may be interconnected in different ways from that shown in FIG. 2. Operation of a computer system such as that shown in FIG. 2 is readily known in the art and is not discussed in detail in this application. Software source and/or object codes to implement the present disclosure may be stored in computer-readable storage media such as one or more of system memory 217, fixed disk 244, optical disk 242, or floppy disk 238. The operating system provided on computer system 210 may be a variety or version of either MS-DOS® (MS-DOS is a registered trademark of Microsoft Corporation of Redmond, Wash.), WINDOWS® (WINDOWS is a registered trademark of Microsoft Corporation of Redmond, Wash.), OS/2® (OS/2 is a registered trademark of International Business Machines Corporation of Armonk, N.Y.), UNIX® (UNIX is a registered trademark of X/Open Company Limited of Reading, United Kingdom), Linux® (Linux is a registered trademark of Linus Torvalds of Portland, Oreg.), or other known or developed operating system. In some embodiments, computer system 210 may take the form of a tablet computer, typically in the form of a large display screen operated by touching the screen. In tablet computer alternative embodiments, the operating system may be iOS® (iOS is a registered trademark of Cisco Systems, Inc. of San Jose, Calif., used under license by Apple Corporation of Cupertino, Calif.), Android® (Android is a trademark of Google Inc. of Mountain View, Calif.), Blackberry® Tablet OS (Blackberry is a registered trademark of Research In Motion of Waterloo, Ontario, Canada), webOS (webOS is a trademark of Hewlett-Packard Development Company, L.P. of Texas), and/or other suitable tablet operating systems.

Moreover, regarding the signals described herein, those skilled in the art recognize that a signal may be directly transmitted from a first block to a second block, or a signal may be modified (e.g., amplified, attenuated, delayed, latched, buffered, inverted, filtered, or otherwise modified) between blocks. Although the signals of the above described embodiments are characterized as transmitted from one block to the next, other embodiments of the present disclosure may include modified signals in place of such directly transmitted signals as long as the informational and/or functional aspect of the signal is transmitted between blocks. To some extent, a signal input at a second block may be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

In several embodiments of the invention, server 110 is associated with a particular location 120. Such a location may include a building, a set of buildings, an area of ground, a stadium or plaza, or other gathering place. Further, the master contact tracing domain may also be implemented on server 110, or optionally on another server shown as master contact tracing domain 130. The location may also enabled with contact tracing equipment, or it may rely on the user device space-time coordinate recording. Such space-time monitoring equipment may be one or more antennas, cameras, proximity sensors, or other sensing equipment. The identify of a mobile device may be determined by an anonymous temporary ID number. The mobile device may then be traced or tracked by WiFi, Bluetooth, cellular, GPS, or other wireless technologies and may also be coordinated with cameras and/or other sensory data. The ability associate a wireless mobile device with a location is well known and not discussed in detail in this disclosure. For purposes of the present disclosure, the minimal requirement is that the user mobile device is enabled to determine its physical space-time coordinate position with a reasonable accuracy and provide that data, if allowed by the user, to the location and/or master contact tracing domain. The embodiments of the invention utilize such equipment that exists in the art and is adaptable to future advances in location determining technology.

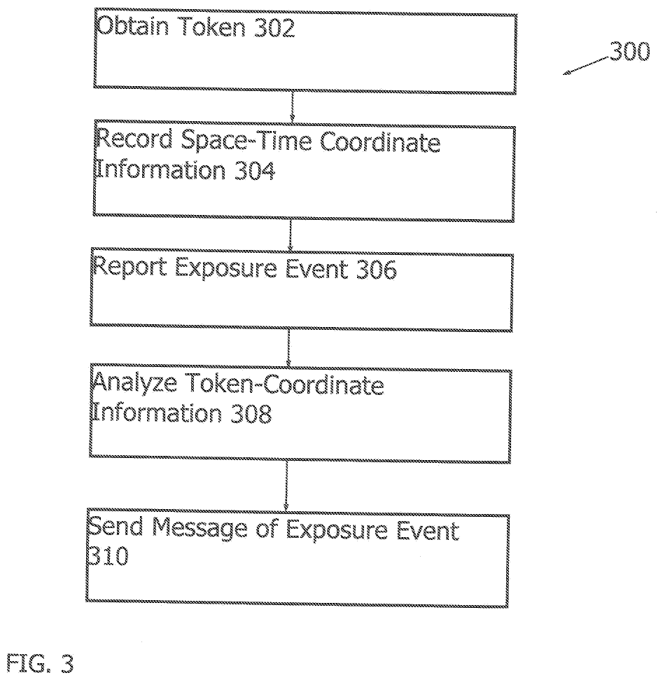

FIG. 3 is a flow chart diagram illustrating operations 300 of one embodiment of the invention. The first step 302 is for the user to obtain a token from a location, for example without limitation using a website in a mobile browser, or an app of the user's mobile device (such as software 116 operating on one of clients 112 of FIG. 1). In this step, the location has a website (such as server 110 of FIG. 1) for visitors to enroll anonymously so that the user would be given a unique token from the location for future tracing. In one embodiment of the invention, this token is stored in the user's local browser storage. In other embodiments, this token is stored in a predetermined location on the user's device. Similarly, the user's space-time information (according to the user's mobile device) is stored in another predetermined location on the user's device, optionally the same location as the token.

In the second step 304, space-time coordinate records are created and associated with each token while in the location, optionally continually created and recorded. The user mobile device maintains the space-time coordinate information for possible future access. In several embodiments, this step involves three parties: server 110 of location 120 operates in conjunction with master contact tracing domain 130. In these embodiments, software 116 of one of clients 112 receives instructions to incorporate a MACT page (with parameters) in the browser memory, which instructions the browser then executes to store the tokens locally in a browser memory associated with the associated MACT domain information.

In a third step 306, the user may report the existence of an exposure event (for example, interacting with an individual having tested positive for a virus, or entering an area with an abnormal concentration of mold or spores) to the location using the user's token and a description of the exposure event.

In a fourth step 308, location 120 and/or master contact tracing domain 130 may analyze all of its token-coordinate information to determine all the tokens that have been subject to the exposure event. Master contact tracing domain 130 may also determine if the reporting user's contacts have impacted other locations, and notify any other locations so that if the master contact tracing domain 130 does not have contact information for other “exposed” tokens, the location server may notify potentially exposed tokens. All token holders may then be sent a warning message indicating the existence of the exposure event in step 310. In one embodiment, the warning message may include an indication of all effected token holders, and the mobile device through the website or the app would check its token value against the token identifications. In another embodiment, the warning message may only indicate that an exposure event occurred and the user device would have the option to check to see if the user's stored token was one of the identified exposed tokens.

In one embodiment, a virtual queuing solution is employed in which a user visits a website to obtain a unique ticket that constitutes a request to access a location. Upon being granted access, the ticket's unique token is marked as activated and thereby constitutes a unique digital access pass to the location. Upon completion of a visit to the location, the pass is cancelled. The combination of the physical location and temporal period of validity of a unique token determines a space-time region. The space-time region is uniquely associated with the user but whilst the time window, physical location and unique token are known to the embodying or local website the identity of the user is not revealed.

In an exemplary embodiment of the invention, the location website and the master contact tracing domain are implemented such that the space-time information is stored in the user's local browser storage. Upon a user inquiring about an exposure event to either the location website or the master contact tracing domain, the website with which the user made the inquiry would then request access to the space-time coordinate history stored on the user device, for example without limitation in the browser storage associated with the master contact tracing domain, and perform a suitable space-time overlap correlation in order to report to the user if they had been exposed.

It will be apparent to those skilled in contact tracing that it is advantageous to maintain a dataset of as many space-time locations as possible in order to maximize the information available for correlation and notification.

One exemplary embodiment of a website that supports the private aggregation in the user's browser of multiple such space-time datasets is now described. The space-time generating website incorporates an iframe (ref) in its page. The “src” attribute of the iframe, indicating the web location to embed into the website's page, is not set. The user interacts with the website and is eventually given an access pass. At this time the website is aware of the essential contact tracing information viz the location, the time and the unique token.

The website sets the “src” attribute of the iframe to a webpage whose URL has a domain such that it is the master contact tracing domain. The location web site and the master contact tracing domain need not be the same, and in several embodiments to enable cross-location tracing several location websites may participate with one master contact tracing domain. Additionally, the website provides the contact tracing information as parameters to the URL of the master contact tracing page. Optionally the contact tracing information in the URL parameters may be encrypted using a public key associated with the location.

A location website may include explanatory explanations of the “Anonymous Contact Tracing” software, in some embodiments termed “OurSafeQ”, with sentences such as “This page uses your local browser storage to maintain a list of OurSafeQ lines you have recently visited.”; “If you ever need to find out if you were close to an infected person while using OurSafeQ you can use this list for anonymous contact tracing.”; and “The OurSafeQ Anonymous Contact Tracing Software is not tracking you. This is your data, stored locally on your device. A history item is retained for a maximum of 4 weeks.”

The act of setting the “src” attribute causes an event in the browser that triggers it to make a web request for the URL (i.e. the master contact tracing site). The page that is loaded from the request gathers the contact tracing information from the URL parameters and, as in the elementary embodiment, it stores the tracing record in the user's local browser storage. Importantly, because the embedded page originated from the master contact tracing domain, the local storage it uses is associated with the master contact tracing domain and not the embodying website, that is the location website. The location website may combine the embedded page from the master contact tracing domain with its own information, the token, and/or the space-time coordinates in the user's browser storage, and may hash or encrypt that data so the master contract tracing domain may only access that information if the location website fetches and decrypts the information.

At no time does the master contact tracing site pass the data to the server of the page. The embedded iframe page that is storing the record is executing in the browser of the user. Indeed the embedded contact tracing page may be cached indefinitely in the user's browser cache and after the first request may not require any future network access, no matter how many embodying location/websites are visited. It will be apparent to those skilled in the art that every time a user visits a location and embodying website the space-time region is recorded locally in the users browser. This applies for any website in any domain provided that they incorporate the master contact tracing page as described. It will be clear to those skilled in the art that this embedded iframe technique preserves anonymity and can ensure all space-time data is only ever held in the user's local browser in at least some embodiments. This principal can be understood as an inverse of certain common embedded-iframe web-tracking techniques that may be used by advertisers and large internet service platforms to track and remove anonymity in regular web browsing across multiple sites.

From a user perspective, in one embodiment, the process starts by the user contacting the location contact tracing system, for example without limitation, visiting a website associated with the location. The location website provides an identification of the location (to distinguish between contact tracing locations) as a queue and the identification may be a queue identification (or QID). The user may request a ticket or token from the location, for example without limitation, by activating the appropriate portion of the location website, and thus obtain a unique ticket (or TID or token). The location website responds to the request by granting admission and sending the token and/or any other access information. For example without limitation, the location web page may contain the embedded iframe src set with parameters on the URL of Time QID and TID. The user mobile device would then fetch instructions on how to interact with the queue at the QID, for example without limitation using a Master Anonymous Contact Tracing page (or MACT) and preforming associated instructions, for example without limitation running a javascript to pull T, QID and TID from URL and store in local storage under MACT domain name. At some point after those initiating events, the location traces or tracks the user mobile device until the user leaves and/or the admission pass expires.

When a user comes to understand that the user is diagnosed with an illness or learns they had been subject to an exposure event prior to visiting a particular location, the user would then be able to report the exposure event to the location. From the perspective of the reporting user, the user would first contact the location, for example without limitation by visiting the location's queue website. At the point of contact with the location, the user declares that it had had an exposure event. The location would then direct the user to the location contact tracing system, for example without limitation the MACT reporting site. The location contract tracing system then obtains the user's time-space coordinate information, for example without limitation the MACT requests local storage log of site visits {{T, QID, TID}, . . . ) from the user mobile device. The location contact tracing system receives this user time-space coordinate information, for example without limitation, by the MACT retrieving a log set from the user mobile device. The location contact tracing system may then analyze the user time-space coordinates to determine if any other users had contact (as may be calculated determined by the exposure event) with the reporting user, for example without limitation by MACT comparing each QID of TID and Time—so the MACT may determine potential exposure risk of each QID, without knowing the identities of any of the QIDs/tickets/tokens.

Once an exposure event is known to a user, for example without limitation, the user receiving a message from a location contact tracing system that an exposure event occurred, the user may check to see if that particular user was exposed. An inquiring user contacts the location contact tracing system, for example without limitation by using the user mobile device to visit the queue website (using the QID), wherein the website offers the user an exposure screening. Once the user accepts the screening the location may direct the user to connect the mobile device to the contact tracing system, for example without limitation by directing the user mobile device to MACT-Screening page (MACTS) on a browser of the mobile device. The location contact tracing system then obtains the user time-space coordinate information, for example without limitation by the MACTS asking the mobile device for the coordinate logs, wherein the MACT correlates the log time-space coordinate information with the contact tracing system tracing records. If the user was exposed, the location contact tracing system informs the user of overlap proximity to its exposure information and may advise the user to take appropriate steps, for example without limitation to be tested for an infectious disease that was the subject of the exposure event.

It will be further apparent that this solution is completely anonymous and that, as there is no use of cookies or other tracking technology, the user cannot be tracked from one location to another. It will be apparent to those skilled in contact tracing that in the event of the need to report infection all that is required is for the user to visit a reporting page on the master contact tracing site and to give permission for the tracing record set to be uploaded. The set can be used to notify the locations of the times at which the reportee was present and to mark these times as potentially infectious. It will be apparent that it is possible, though not necessarily desirable, to report infection and retain anonymity. It will be apparent to those skilled in contact tracing that any location knowing of a time of infection may present a notice to users directing them to a screening site at the master contact tracing site. As with reporting, the screening page may request for the user's tracing record to be used to determine if they were present at any location at a time when they might have been exposed. It should be noted that this screening is anonymous. It will be apparent to those skilled in the art that the master contact tracing site might ask the user if they wish to be actively notified.

Any user wishing to be actively notified simply supplies contact information (phone, email, etc.) and gives consent for their location record to be checked each time a new record is stored. The master site stores this contact information in the user's browser along with the contact tracing information. Every time the user visits a location performing contact tracing via the master site they may be notified of any exposure at any of their previous visits. The contact information is essentially held in escrow in the users own browser and is only needed if a notification is required. Hence this solution is also anonymous.

It will be apparent to those skilled in the art that a further embodiment might request that the user's contact and history be stored with the master tracing system in two separate systems. Any report of infection of a location that is correlated with the users space-time history would be the necessary condition to access the user's contact information and to perform a notification to the user of the historical risk of infection. It will be apparent that the user remains anonymous until such time as a risk of exposure is identified and notification contact is made.

While one or more embodiments of this invention have been described as having an illustrative design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A method for anonymous contact tracing for a user with a mobile device in a location having an associated contract tracing system having a plurality of tokens, comprising the steps of:
obtaining one of the plurality of tokens from one of the location or the contact tracing system using a user mobile device;
storing the user's space-time coordinate information on the mobile device;
recording space-time coordinate information of each mobile device that is traceable by the contact tracing system in association with one of the tokens;
reporting the existence of an exposure event by the user;
analyzing the token-coordinate information to determine which of the plurality of tokens have been subject to the exposure event; and
notifying at least one of the users with an associated token that was subject to the exposure event.

2. The method of claim 1 wherein the contact tracing system includes a server separate from and in communication with the location.

3. The method of claim 2 wherein the step of obtaining is performed by the mobile device requesting a token from one of the location or the contract tracing system server.

4. The method of claim 1 wherein the token is uniquely identifiable by at least one of the location or the contact tracing system, and the token lacks any personally identifying information relating to the user.

5. The method of claim 1 wherein the obtained token is stored with the user's space-time coordinate information.

6. The method of claim 1 wherein the token and the user's space-time coordinate information are stored in a browser storage of the mobile device.

7. The method of claim 1 wherein the reporting includes the user identifying the user's token and a description of the exposure event.

8. The method of claim 1 wherein the notifying step includes sending a message with an indication of tokens subject to the exposure event.

9. The method of claim 8 further including the step of the user's mobile device checking the mobile device's stored token value against the token identifications of the message.

10. The method of claim 1 wherein the notifying step includes sending a warning message indicating that an exposure event occurred.

11. The method of claim 10 wherein the user's mobile device contacts at least one of the location or the contact tracing system to determine if the token stored on the user's mobile device was identified as an exposed token.

12. A server for performing anonymous contact tracing, the server being in communication with at least one location, the server having a plurality of tokens, the server comprising:
a processor and associated memory;
a communications module configured for communication with mobile devices; and
software for performing the steps of:
providing a first token of the plurality of tokens to a first mobile device of a first user;
providing a second token of the plurality of tokens to a second mobile device of a second user;
enabling each of the first and second mobile devices to record space-time coordinate information of the associated user with an associated token;
receiving a report of the existence of an exposure event by one of the first and second user;
analyzing token-coordinate information to determine tokens that have been subject to the exposure event; and
notifying the other of the users if an associated token is determined to have been subject to the exposure event.

13. The server of claim 12 wherein the software performs the providing step via a website of the at least one location.

14. The server of claim 12 wherein the token is uniquely identifiable by the contact tracing system, and lacks any personally identifying information relating to the user.

15. The server of claim 12 wherein the software instructs the mobile device to store the token, the user's space-time coordinate information, and domain information of the server, in a browser storage of the mobile device.

16. The server of claim 12 wherein the notifying step includes sending a message with an indication of all affected token holders.

17. The server of claim 12 wherein the notifying step includes sending a warning message indicating that an exposure event occurred.

18. The server of claim 17 with the software further comprising the step of responding to an inquiry from a user mobile device to determine if the token from the user mobile device was one of the exposed tokens.

19. An App operating on a user mobile device for anonymous contact tracing for the user, the mobile device having a processor and associated memory, the App configured to communicate with a location in communication with a contract tracing system with a plurality of tokens, the App having software enabling the mobile device to perform the steps of:
sending a request to obtain one of the plurality tokens from the contact tracing system;
receiving and storing the token on the mobile device;
storing the user's space-time coordinate information on the mobile device;
reporting the existence of an exposure event to the location by the user;
analyzing space-time coordinate information to determine if the mobile device token had been subject to the exposure event.

20. The App of claim 19, wherein the software enables a further step of storing token information, the user's space-time coordinate information, and domain information of the server, in a browser memory location.
